(19) United States
(12) Patent Application Publication
(10) Pub. No.: US 2025/0345023 A1
(43) Pub. Date: Nov. 13, 2025

(54) SYSTEM FOR PERFORMING A SOUND-BASED SENSING OF A SUBJECT IN A SENSING AREA

(71) Applicant: SIGNIFY HOLDING B.V., EINDHOVEN (NL)

(72) Inventors: JIN YU, LEXINGTON, MA (US); PETER DEIXLER, ARLINGTON, MA (US)

(21) Appl. No.: 18/854,119

(22) PCT Filed: Mar. 29, 2023
(86) PCT No.: PCT/EP2023/058092

§ 371 Date: Oct. 4, 2024

Related U.S. Application Data
(60) Provisional application No. 63/326,957, filed on Apr. 4, 2022.

(30) Foreign Application Priority Data
Apr. 14, 2022 (EP) 22168412.9

(51) Int. Cl. A61B 7/00; A61B 5/00; H04R 1/40; H04R 3/00; H04R 3/04

(52) U.S. Cl. CPC A61B 7/006; A61B 5/0022; A61B 5/4561; H04R 1/406; H04R 3/005; H04R 3/04

(57) ABSTRACT

A system for providing a sound-based sensing of subjects comprising a network (10) of network devices (100) configured to perform a sound-based sensing. The network devices (100) are distributed in a sensing area (20) and communicate with each other based on RF signals. At least one network device (100) comprises a sound generator (120) to generate a sound signal (100) and/or a sound sensor (130) to detect the sound signal (101) from the sound generator (120) after a propagation through at least a portion of the sensing area (20) and to generate a sensing signal indicative of the detected sound signal. The sound generator (120) and the sound sensor (130) are arranged in different network devices (100). A sitting position detector (200) detects a sitting position of a subject in a sensing area based on the sensing signal from the sound sensor (130).

### FIELD OF THE INVENTION

The invention relates to a system, a method and a computer program for performing a sound-based sensing of a subject in a sensing area.

### BACKGROUND OF THE INVENTION

As many people are working on a computer and are sitting at a desk, a sitting position of the person is very important in view of improving the health of the person. Non-ergonomic sitting positions can lead to a decreasing health in particular in view of back pains. It should be noted that improper sitting posture at home or at the office may not lead to an incent injury but the injury may develop over months or years if a prolonged exposure to improper posture is present.

FIG. 1 shows a representation of a healthy sitting position at a desk. A person 30 is sitting on a chair 40 at a desk 40 and is typing on a keyboard 42 and watching at a display 41. Further, a lamp 34 is placed on the desk 40. For an ergonomic sitting posture, a lumbar angle 4 should be ≤20°, a cervical angle 3 should be ≤20°, a sight angle 2 should be between 15 and 30° and a sight distance 1 should be between 50 and 80 cm. A prevention of improper sitting posture is very important to improve the health of people often sitting at a desk.

Several camera based or pressure sensor based solutions for detecting an improper sitting posture are known.

“Sitsen: Passive sitting posture sensing based on wireless devices” by Li et al, International Journal of Distributed Sensor Networks, Jul. 7, 2021 shows a non-contact wireless-based sitting posture detection using RFID tags.

US 2012/116252A1 discloses detection of body orientation and/or posture. At least one wave sensor may be configured to output waves and collect measurements data based upon the reflections of the output waves. At least one processor may be configured to receive measurements data from the at least one wave sensor and evaluate the received measurements data to determine a posture of a monitored subject. Based at least in part upon the determined posture, one or more suitable control actions may be implemented.

### SUMMARY OF THE INVENTION

It is an object of the invention to provide a non-obtrusive sensing capability enabling a detection of improper sitting posture of a subject.

In a first aspect of the present invention a system for performing a sound-based sensing of subjects in a sensing area is presented. The sensing is performed by a network of network devices performing a sound-based sensing in the sensing area. At least one network device comprises a sound generator and at least one network device comprising a sound detector. The network devices are distributed in the sensing area and communicate with each other based on RF signals. The sound generator generates a sound signal and the sound detectors detect the sound signal after a propagation through at least a portion of the sensing area. The sound sensor generates a sensing signal indicative of the detected sound.

The sound generator and the sound sensor are arranged at or in different network devices. A sitting position detector detects a sitting position of the subject based on the sensing signal. Hence, an active detection can be provided that allows for actively sensing a subject in the sensing area.

The network of network devices which can also be understood as a sensing network comprises at least two network devices, in particular, at least one network device which is adapted to generate a sound and at least two network devices that are adapted to detect a sound. Preferably, the network comprises more than three network devices, wherein the number of network devices in the network can be adapted based on the sensing area in which a sensing should take place. For example, the larger the space the more network devices can be provided in the network and/or the more complex a shape of the space the more network devices can be provided in the network. Preferably, all network devices comprise a sound generator and are thus adapted to generate sound, and a sound detector and are thus adapted to detect sound. However, the network can also comprise one or more network devices that are dedicated to generate a sound and thus only comprise a sound generator and two or more network devices that are dedicated to detect a sound and thus comprise only a sound detector.

The sitting position detector can be adapted to control the at least one sound generator, in particular, to control a network device comprising the sound generator, to generate a predetermined sound. The predetermined sound can generally refer to any sound that can be provided by the sound generator and comprises a predetermined characteristic like a predetermined length, a predetermined frequency spectrum, a predetermined amplitude, etc.

In an embodiment, the sitting position detector can be configured to estimate a hip and a head position of the subject (in particular a person) sitting at a table or desk. With the information regarding the hip and/or head position, the sitting position detector can perform a better estimation on the sitting position of a person.

In an embodiment, the sitting position detector can comprise a controller to control the operation of at least one of the network devices. Hence, for example, the operation of a sound generator and/or a sound detector in one of the network devices can be controlled by the sitting position detector.

In an embodiment, the controller of the sitting position detector can control the operation of the sound generator to determine a sound wave and/or a frequency of the generated sound signal in the at least one first and/or second audio channel. Accordingly, the sitting position detector can control the operation of the sound generator. Optionally, the controller can adapt the operation of the sound generator if required.

In an embodiment, the first audio channel can be in an audible frequency range and the second audio channel can be in an inaudible frequency range. Optionally, the audible frequency range can be below 18 kHz. Optionally, the inaudible frequency range can be >18 kHz. It is thus also possible that a sound signal is generated which is not audible by a human and does thus not negatively influence the human. The frequency ranges can also be adapted if pets are present. Optionally, the user of the system may provide information to the controller whether or not pets may be present or whether other restrictions may occur or are requested.

In an embodiment, the at least one sound generator generates a sound signal in a first audio channel. An at least one second generator generates a second sound signal in a second audio channel. An at least one sound sensor is configured to detect a sound signal in the first audio channel and to generate a sensing signal indicative of the detected sound signal. The sound sensor can also be configured to detect sound signals in the second audio channel and to generate a sensing signal indicative of the detected sound signal. As an alternative, a second sound sensor may be provided for the second audio channel. Thus, the sound based detection can be performed based on sound signals with different frequencies to increase the accuracy and robustness of the sound based sensing of subjects.

In an embodiment, the controller of the sitting position detector can control the sound generator generating an audible sound signal or an inaudible sound signal in the first audio channel and can control the sound generator generating the inaudible sound signal or the audible sound signal in the second audio channel. The controller can optionally initiate the generation of the inaudible sound signal or the audible sound signal only when the sitting position detector has detected a correct head position of the subject. Hence, it is possible to reduce the amount of emitted sound signals in the sensing area. If a detected head position is not a correct head position, then the detection of the hip position can be avoided. Thus, audio pollution levels can be reduced in the system.

In an embodiment, the sitting position detector comprises a fall detector to determine whether a subject or person has fallen off a chair based on the sensing signal from the sound sensor. The system can thus be extended to a fall detection. This can in particular be advantageous if the person sitting at the desk are older people or if the people working at the desk are prone to falling asleep.

In an embodiment, the sitting position detector can determine an abnormality regarding sitting posture compared to previous determined sitting postures based on the sensing signal from the at least one sound sensor and issues a warning if an abnormability is determined. This is also advantageous for detecting or monitoring for example elderly people to warn regarding any sitting postures which may be indicative of an illness or sickness.

In a further aspect of the invention, a method for performing a sound based sensing of subjects in a sensing area based on a network of network devices configured to perform a sound based sensing in the sensing area is provided. The network devices are distributed in the sensing area and are configured to communicate with each other based on RF signals. A sound signal is generated by a sound generator in one of the network devices. The sound signal from the sound generator is detected after a propagation through at least a portion of the sensing area and a sensing signal indicative of the detected sound by the sound sensor in one of the network devices is generated. The sound generator and the sound sensor are arranged at different network devices. A sitting position of a subject in the sensing area is detected based on the sensing signal from the sound sensor.

In an embodiment, the absolute sitting position or a relative sitting position of a user can be detected. The relative sitting position can be used to determine abnormabilities in the sitting posture for example of elderly people. A slump posture may indicate a diagnostic feature of depression.

According to an embodiment, a first head position is detected with an inaudible sound sensing (which has only a short sensing range). This can be directed to detecting a head position. If a correct head position is detected, an audible sound sensing (which has a long sensing range up to 5 m) can be activated.

Alternatively, first an audible sensing is performed to determine a torso position (hip position) with a long sensing range (audible audio signals). Only if the torso position is ok, an inaudible sensing is performed to detect the head position.

According to an embodiment, a smart device can be used to forward information to a user on how to position the smart device to improve the audio sensing. For example, a first network device can be implemented as a lighting device with a sound generator and for example a microphone of a smart device like a laptop or smartphone can be used as sound sensor in the system.

The sitting position detector can comprise a controller which can control the operation of the network devices. In particular, the controller can control the operation (e.g. the sound waveform, the frequency of the sound signal) of the sound generators. The sitting position detector may also comprise a memory to store and track the detected positions of the head and/or the hip over time. The sitting position detector may also comprise a fall detector which is able to detect a fall of the person from a chair based on the detected audio signals from the sound sensors. The sitting position detector may also comprise an alert unit for initiating an alert to the person if an improper sitting position or if a fall is detected.

Generally, a sound generator and a sound detector can be regarded as a sound propagation pair between which the sound generated by the sound generator propagates in a multipath transmission channel from the sound generator to the sound detector. Preferably, the sound generator and the sound sensor are arranged at or in different network devices. An audio multipath transmission channel can be, for instance, three-dimensional and shaped such that it is narrow at the point of the sound generation, wide during the propagation through the space and again narrow at the point of the detection by the sound detector. However, the exact shape of an audio multipath transmission channel is determined by the environment, in particular, the sensing area, through which the sound propagates. Generally, an audio multipath transmission channel can be regarded as comprising multiple audio paths due to the different reflections of the generated sound on one or more surfaces. For instance, one of the multiple audio paths may refer to the sound being reflected by a table before detection, one may refer to a direct path between the generation and the detection, and one may refer to the reflection from a wall before the detection. Since the sound is generated by at least one sound generator and then detected by a plurality of sound detectors, different, i.e. multiple, channels comprising again multiple audio paths can be exploited during the sensing. Thus, the propagation of the sound after the generation refers to a multi-channel propagation, wherein each detector detects one of the multi-channels as detected sound. Based on this detected sound, the sitting position detector is then adapted to control the detectors to generate a sensing signal that is indicative of the detected sound. The sensing signal can refer, in particular, to an electrical signal that is indicative of the detected sound.

The sensing signals utilized by the subject determination unit are indicative of the detected sound during a predetermined time period of, for instance, a few seconds. Thus, the sensing signals refer generally not to one value, but to a continuous measurement of the detected sound during the predetermined time period. The time period can, for instance, be determined by the length of the predetermined sound that is generated by the sound generator and the measurement of the detected sound can start, for instance, at the same time at which the sound generator starts to generate a predetermined sound. In particular, in small sensing areas the travel time of the sound between the sound generator and the sound detector can be neglected.

In an embodiment, the status and/or position is determined based on i) the signal strength of the plurality of detected sensing signals and/or based on ii) channel state information derived from the plurality of detected sensing signals and the predetermined generated sound. With respect to the first option, a status and/or position of at least one subject in the sensing area can be based on the signal strength of the plurality of detected sensing signals. Since a signal strength of the sensing signal depends on an amplitude of the sound that directly or indirectly reaches the sound detector, the single strength is indicative of the different paths the sound can travel from the sound generator to the sound detector. Since these paths are highly dependent on the environment between the sound generator and the sound detector and thus also dependent on the position of subjects in the environment, the signal strength is indicative of these positions. Moreover, since the signal strength of a plurality of detected sensing signals is utilized, wherein the sensing signals result from the detection of sound detectors arranged at different locations, information on the environment of the network devices from a plurality of different sound paths is provided by the sensing signals.

In the second option, the position of at least one subject in the sensing area can be based on channel state information derived from the plurality of detected sensing signals and the determined generated sound. The channel state information is indicative of the properties of a path that the sound has taken from the sound generator to the sound detector and thus describes how the sound has propagated from the sound generator to the sound detector. Accordingly, the channel state information is also indicative of an interaction of the sound with the subject along the propagation path. Thus, the channel state information provides very accurate information on the environment of the network with which the sound has interacted, for instance, from which it has been reflected, scattered or absorbed. Since the predetermined generated sound is known and due to the network characteristics of the network, the channel state information can be derived from the sensing signals and the predetermined generated sound.

In an embodiment, the sitting position detector is adapted such that the sound generator generates the predetermined sound as a directed sound, wherein the directed sound is directed to the at least one person. Generating a directed sound has the advantage that the influence of other subjects that should not be detected can be minimized. Moreover, also the influence of the general environment like, for instance, walls, a ceiling or a floor on the detected sound can be minimized. If a direct line of sight from the sound generator to the person is obstructed, the directed sound can also be directed to a flat surface in the room such that the reflection of the flat surface reaches the subject. In such an embodiment, it is preferred that the flat surface does not often change its status and position such that a change in the sensing signal is only indicative of a change of the subject and not a change of the flat surface that also lies in the signal path. For generating the directed sound any known methods can be employed. For example, the sound generator can be adapted to comprise a speaker array with a plurality of sound generator speakers that allow to direct the sound generated by the speaker array based on an interference of the sound generated by each individual speaker.

In an embodiment, the sitting position detector is adapted such that the sound generator generates the predetermined sound as an omnidirectional sound. Generating the predetermined sound as omnidirectional sound has the advantage that a status and/or position of the whole environment of the network can be taken into account.

In an embodiment, each sound detector comprises a sound detector array such that the plurality of sensing signals are each indicative of a direction from which the detected sound has reached the detection array, wherein the sitting position detector is adapted to determine the status of the person further based on the direction information provided by each sensing signal. In particular, a sound detector array allows to more accurately determine which path the sound has propagated from the sound generator to the sound detector and, in particular, to differentiate between these different paths. This allows for a more accurate determination of the status and/or position of an object. In particular, when determining the status of a person in the space using the direction information can be advantageous.

In an embodiment, each network device comprises a sound detector and a sound generator. The sitting position detector is adapted to control the sound generators of the network devices to generate a predetermined sound and the sound detectors of all other network devices to detect the generated sounds such that for each sound generated by different sound generators a plurality of detected sensing signals are generated, wherein the position of the person is determined based on each of the plurality of audio sensing signals.

In an embodiment, the sitting position detector is adapted to control the sound generators of the network devices to subsequently generate a predetermined sound and the sound detectors of all other network devices to detect the subsequently generated sounds. In particular, the sitting position detector can be adapted to control a first network device, i.e. sound generator of the first network device, to generate a predetermined sound and to control all other network devices, i.e. the detectors of all other network devices, to detect the generated sound of the first network device to generate a sensing signal corresponding to the first generated predetermined sound. Then, the sitting position detector is adapted to control a second network device to generate a predetermined sound and all other network devices to detect the predetermined sound to generate the sensing signals that correspond to the second generated predetermined sound and so on until all network devices have at least once generated a predetermined sound. The sitting position detector is then adapted to determine a position of the at least one person in space based on all sensing signals, wherein also in this case, for example, the already above described methods for determining the status and/or position of the subject based on each of the plurality of audio sensing signals can be utilized. The time series of different predetermined sounds generated by different sound generators can be similar or can be different to each other.

In another preferred embodiment, the sitting position detector is adapted to control the sound generators to generate different predetermined sounds concurrently and the sound detectors of all other network devices to detect the different generated sounds such that a sensing signal for each different predetermined sound is generated by the sound detectors.

The different predetermined sounds preferably refer to sounds lying within different frequency ranges. For example, the sitting position detector can be adapted to control a first sound generator to generate a first sound with a first frequency in an audible frequency range, and at the same time a second sound generator to generate a second sound with a second frequency in an audible frequency range. If the first and second frequency are chosen to lie within sufficiently different frequency ranges, the two detected sounds can be separated by the sound detectors to generate different sensing signals for the different predetermined sounds. Thus, sensing signals referring to different combinations of sound generators and sound detectors can be sensed at the same time. Moreover, the above described embodiments can also be combined.

In such an embodiment, the sitting position detector is adapted to control the sound generators of the network devices to subsequently generate different predetermined sounds and the sound detectors of all other network devices to detect the subsequently generated different sounds. In particular, in situations in which for different combinations of sound detectors and sound generators different frequencies are of interest for determining a status and/or position of a subject, such a combination can be advantageous. According to an embodiment, the network devices can be implemented as light (ceiling light, desk lamp), as smart device (smartphone, smart watch, tablet, smart speaker) or as network capable electronic device (e.g. laptop). In an embodiment, at least one of the network devices comprises a lighting unit to implement a lighting functionality. However, in other embodiments the network devices can also comprise other functionalities like entertainment functionalities, monitoring functionalities, etc.

In another aspect of the invention, a computer program product for performing a sound based sensing of a person in a sensing area is presented, wherein the computer program product comprises program code means for causing the system as described above to execute the method as described above.

It shall be understood that the system, the method, the computer program, and the network comprising this system, have similar and/or identical preferred embodiments, in particular, as defined in the dependent claims.

It shall be understood that a preferred embodiment of the present invention can also be any combination of the dependent claims or above embodiments with the respective independent claim.

It shall be understood that the aspects described above and specifically the system of claim 1, the method of claim 14 and the computer program product of claim 15 have similar and/or identical preferred embodiments, in particular as defined in the dependent claims.

These and other aspects or embodiments of the invention will be apparent from and elucidated with reference to the embodiments described hereinafter.

### BRIEF DESCRIPTION OF THE DRAWINGS

In the following drawings:

FIG. 1 shows a representation of a healthy sitting position at a desk,

FIG. 2 shows a block diagram of a system for performing a sound based sensing of a person in a sensing area,

FIG. 3A, 3B and 3C each show a representation of a sound based sensing of a person at a desk, and

FIG. 4 shows a flow chart of a method of sound based sensing of a person in a sensing area.

### DETAILED DESCRIPTION OF EMBODIMENTS

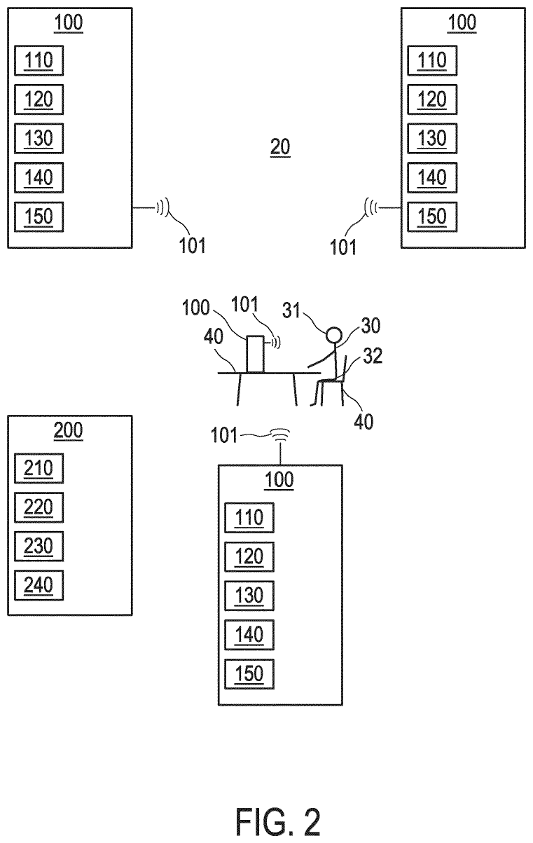

FIG. 2 shows a block diagram of a system for performing a sound based sensing of a person in a sensing area. A sensing system comprises a network 10 of preferably several network devices 100. The network devices 100 are able to communicate with each other based on RF signals. A network device 100 comprises an RF transceiver 110 or an RF transmitter as well as an RF receiver for the RF communication. A network device 100 can comprise a sound generator 120 and/or a sound detector 130. The sound generator 120 can be implemented as a speaker which is able to generate directional or omnidirectional sound. The sound sensor 130 can be implemented as a microphone or a microphone array. Optionally, the sound sensors 130 of the several network devices 100 can be used as a microphone array. Here, the detected sound can be processed and the position information of the various network devices can be used during the sound processing.

The network device 100 can optionally comprise a lighting unit 140. Furthermore, a network device may comprise a controller 150.

The network devices 100 can be arranged at different positions in a sensing area 20. In the sensing area, a person 30 (e.g. a subject) can for example sit at a desk 40 on a chair 40. The person 30 can have a sitting posture which is characterized by a head position 31 and a hip position 32.

At least one sound generator 120 of one of the network devices 100 generates a sound which propagates through the sensing area 20 and is influenced by the objects 40 and the person 30. The reflected sounds (i.e. the audio multi path transmission) can be picked up by at least one of the sound sensors 130. Based on the received (reflected) sound, a sitting position detector 200 can determine a sitting position of the person 30.

According to an example, multiple audio sensing channels can be used to determine a 3D position estimation of a person 30 in the sensing area 20. In particular, the head position 31 and hip position 32 are detected.

The sitting position detector 200 comprises a controller 210, which can control the operation of the network devices 100. In particular, the controller 210 can control the operation (e.g. the sound waveform, the frequency of the sound signal) of the sound generators 120. The sitting position detector 200 may also comprise a memory 220 to store and track the detected positions of the head 31 and/or the hip 32 over time. The sitting position detector 200 may also comprise a fall detector 230, which is able to detect a fall of the person from the chair 40 based on the detected audio signals from the sound sensors 130. The sitting position detector 200 may also comprise an alert unit 240 for initiating an alert to the person if an improper sitting position or if a fall is detected.

The memory 220 can be used to track historic data of the head 31 and/or hip position 32 of a user. This historical data may also be used by the sitting position detector 200 to estimate a current head and hip position.

According to an embodiment, the sitting position detector 200 can collect the output of the sound sensors 130 over time to track movements as well as the body posture over time. The sitting detector 200 can use this information to perform a longterm analysis of the sitting posture or changes in the sitting posture. In particular, the sitting position detector can perform a fall detection (e.g. determine whether the person has fallen from the chair). Preferably, at least two network devices 100 are used to generate sound and to detect sound to be able to improve the accuracy of the audio based sensing of the person 30. Optionally, one network device 100 can be place on the table 40 adjacent to the user 30. This network device 100 can be implemented or may comprise a lighting unit and function as a desk lamp. Accordingly, the network devices 100 arranged at different positions in the sensing area 20 as well as a network device 100 implemented as a desk lamp can be used to generate sound (audible or inaudible) and detect the reflected sound to enable an audio based sensing of a position of the person 30.

According to an embodiment, a network device 100 can be implemented as a smart device on which an application is running. Here, such a smart device comprises a microphone and a speaker and can be used to generate sound and/or to detect emitted sound. The results thereof can be used by the sitting position detector 200 to determine a sitting position of a person 30. If one of the network devices 100 is implemented as a smart device like a smart phone, the internal sensors of the smartphone which generate orientation data can be used to instruct a user to change the position and/or orientation of the smartphone to enable an improved sound generation or sound receiving. If the smartphone has a touchscreen, the touchscreen can be used as a user interface. If the smart device is implemented as a laptop, user instructions may be displayed on the laptop to change the position and/or orientation of the laptop to improve the sound generation or the sound reception to improve the accuracy of the sitting position detection. In an example, the user instructions may be displayed on the one of the network devices 100. In this example, at least one network device 100 has appropriate screen to display the user instructions.

According to an embodiment, if a network device 100 is implemented as a table lamp, a user may receive audible and/or video information on how to position the network device to improve the accuracy of the sitting position detection.

According to an embodiment, the network devices 100 can be implemented as ceiling lights. A first audible sensing channel between the two ceiling lights may be established to estimate a shape and position of a desk 40 in the sensing area 20. When the first audio sensing channel has localized the desk surface and the chair position, a second inaudible audio sensing channel for example formed between the ceiling lamps 100 and a network device 100 implemented as a table lamp can be used. Based on the information from this second audio sensing channel, the 3D positions of the head 31 of the person as well as a hip position 32 (pressure point between the body and the chair surface) can be estimated and tracked. Based on the head position 31 and the pressure point position 32 (hip position), the sitting position detector 200 can estimate whether the position of the person relates to a healthy or improper sitting position.

According to an embodiment, the first audible sound emitted by a sound generator 120 of one of the network devices 100 (for example implemented as ceiling lights) may be used to detect a head position 31. The accuracy of such a sensing can be >1 m. A second inaudible sound can be emitted by one of the sound generators 120 (the network device implemented as a table lamp). This second audio channel can enable an accuracy for head position estimation in the range of cm. The hip position (pressure points) may be determined based on a combination of the signals from the first and second audio sensing channel. The frequencies for the first and second audio channel must take into account possible interferences from third party devices.

According to an embodiment, the system may also be able to detect several persons sitting in the sensing area and determine their head and hip position to determine whether their sitting position is adequate or not.

In order to minimize any audible disturbances or ultrasonic sound pressures to the user's ear or animals in the sensing area, the first and second sensing channel can be activated subsequently. For example, the second audible audio sensing channel is only activated when the first audible audio sensing channel has determined that a head posture of a person is correct. Then the second inaudible audio sensing channel is actuated to determine the hip position of a user. If the information from the first audio sensing channel indicates that the head posture is not correct, no further audio sensing is performed as a bad head posture will inevitably lead to a bad sitting position. Optionally, the sitting position detector 200 can activate a bad sitting position alert to the user. This can be performed as an audio signal, via a computer on which the user is working or via a smartphone of the user. Alternatively, the alert may also be an optical alert for example on and off switching of the desk lamp or ceiling lamps. The alert may also be a combination of the above.

A network device according to an embodiment can be regarded as any device adapted to form a network with other network devices. In particular, a network device comprises a network device communicator that is adapted to receive and transmit wired or wireless signals, for instance, radiofrequency signals, infrared signals, electrical signals, etc. The network between the network devices can then be formed through a communication between the network devices following a known network communication protocol like WiFi, ZigBee, Bluetooth, etc. Preferably, the network devices refer to smart devices, i.e. devices comprising a communication unit for receiving and transmitting network communication signals but which otherwise fulfil the function of a corresponding conventional device. In particular, such a smart device can be a smart home or office device, in which case the corresponding conventional function would be that of a conventional home or office device. Preferably, the conventional function refers to a lighting function and the network devices refer to network lighting devices that are further adapted to comprise a sound generator and/or a sound detector. However, the network devices can also refer, for instance, to smart plugs, smart switches, etc.

The system can be part of the network, for instance, can be part of one or more of the network devices. In particular, the system can be provided as hard-and/or software as part of one of the network devices or distributed over a plurality of the network devices that are in communication with each other to form the system. However, the system can also be provided as a standalone system, for instance, in a device that is not part of the network of network devices but is directly or indirectly in communication with at least one of the network devices, for instance, to control the network devices. For instance, the system can be provided as part of a handheld computational device like a smartphone, a tablet computer, a laptop etc. However, the system can also be located in a cloud formed by one or more servers, wherein in this case the system might communicate with the network, in particular, the network devices, via one or more devices that are connected to the cloud like a router.

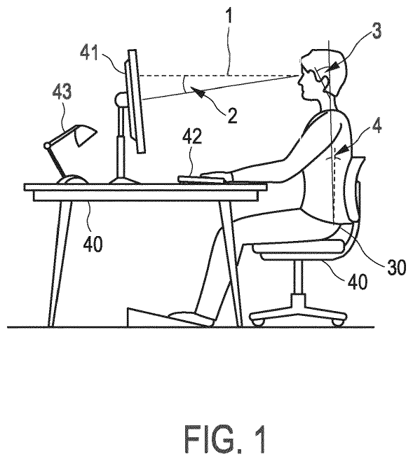

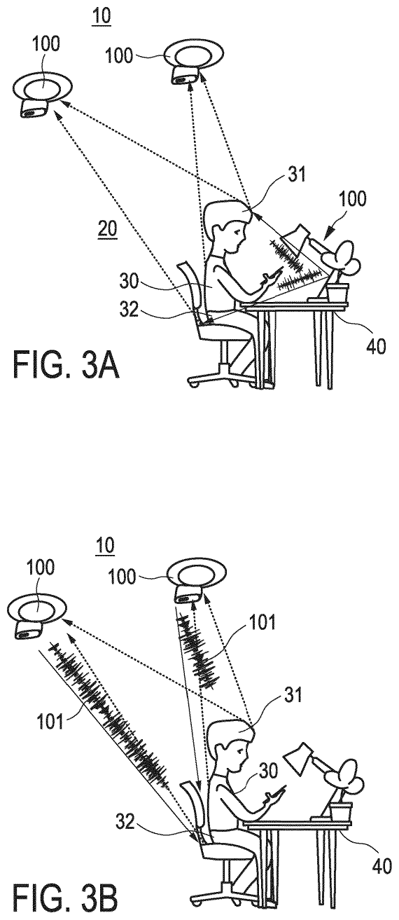

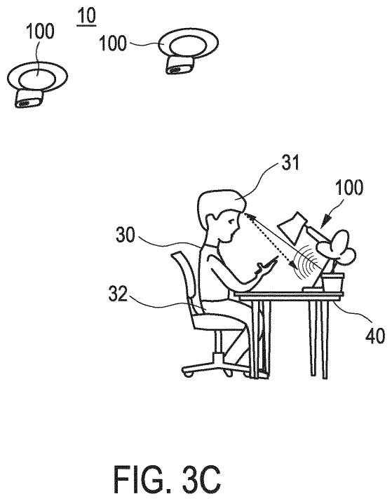

FIG. 3A, 3B and 3C each show a representation of a sound based sensing of a person at a desk. The network 10 may comprise three network devices 100. Two of the network devices 100 may be implemented as ceiling lamps, while a third network device 100 can be implemented as a desk lamp or a smart device placed on a table near a person 30. In the sensing area 20, a person 30 is sitting at a desk 40 on top of which the third network device 100 (table lamp) is placed. The network devices 100 can be implemented or may comprise lighting units. The network devices 100 according to FIG. 3 may correspond to the network devices according to FIG. 1. Therefore, the network devices 300 may comprise sound generators 120 and/or sound detectors 130. The frequency of the sound generated by the sound generators 120 in the ceiling network devices 100 may be different from the frequency of the sound generated by the sound generator 120 in the network device 100 implemented as a table lamp. In particular, the frequency of the sound generated by the sound generator 120 of the table lamp 100 is preferably in the inaudible sound range. Based on the reflected sounds picked up by the sound sensor 130 in the table lamp network device 100, the position of a head 31 of the person can be detected. The inaudible sound based sensing can be performed if the distance between the network device and the head 31 of the user is smaller than 1 m.

Accordingly, the (table lamp) network device 100 emits first sound at a first frequency range which corresponds to an inaudible frequency range. Based on the detected (reflected) inaudible audio signals, the head position 31 as well as the chair position based on the multipath characteristics of the inaudible sensing signal is estimated by a sitting position detector 200.

The ceiling lamp network devices 100 are arranged at a distance from the person 300 which is typically larger than one meter. Thus, the distance is too large for an inaudible sound sensing. Therefore, the ceiling network devices 100 are emitting a second audible sensing signal. The reflected audio sensing signal can be received by a second ceiling network device 100 or by the ceiling network device which emitted the second audible sensing signal. Based on the received audible sensing signal, the shape and position of the desk surface as well as the orientation of the desk with respect to the room's building structure and other objects inside the sensing area is estimated by the sitting position detector 200.

In FIG. 3A the head position 31 of the person 30 is determined by a (desk lamp) network device 100. In FIG. 3B a further position detection is performed by the ceiling network devices 100.

According to an embodiment, the sitting position of a person 30 is detected using a first audio channel based on inaudible sound as well as at least one second audio channel based on audible sound. The first audio channel can be provided by a network device 100 (such as a desk lamp) placed near (<1 m) the person. The second audio channel can be implemented by network devices 100 arranged at a greater distance. Such network devices can for example be ceiling network devices (ceiling light units). The network devices for the second audio channel can also be light units arranged at walls in the sensing area.

According to a further embodiment, the first audio channel with the inaudible sound can be implemented by a smart device or a laptop arranged in the vicinity of the person 30 whose sitting position is to be detected and evaluated.

According to an embodiment, the sound sensor in the first and/or second audio channel can be the same device as the transmitter. Alternatively, a sound generator (transmitter) may be arranged in a different network device than the transmitter emitting the inaudible audio signals in the first audio channel. Moreover, optionally the transmitter transmitting the audible audio signals in or for the second audio channel can be arranged in the same network device as the detector (microphone) for the audio signals. Alternatively, the sound detector may be arranged in a network device different from the network device where the sound generator for the second audio channel is arranged. As an example, the sound sensor may be implemented as a microphone array, where the sound detectors of several network devices are used to detect the audible sound for the second audio channel. This is advantageous as it will allow a more accurate sensing of the head position 31 and hip position 32 of the person 30.

In an embodiment, sound generators from different network devices may be used in combination to generate and emit an audible sound in the second audio channel. The frequencies of the sound signals generated by the respective sound generators may be different for the different network devices.

According to a further aspect of the invention, in a first step, the approximate position of the person or subject 30 in the sensing area is detected and then a more exact position detection may be performed, wherein the frequencies at which the sound generators generate the sound signals can vary in time or generate different frequencies for the different network devices (the frequency may depend on the position of the network devices 100 in the sensing area 20). The approximate and/or exact location/position of the subject or person 30, in an example, may be determined by RF-based sensing. The position of the network device 100 may be with respect to the position of the subject 30. This can lead to a more exact determining of the head and/or hip position of the user to allow an estimation of the sitting position of the user.

By using the first audio channel (inaudible audio signal), it is possible to track the head and the hip position of the person relative to the deck chair furniture at a cm level. Based on the 3D head position 31 and hip position 32, the sitting position detector 200 can determine a healthiness score associated to the various positions of the head and hip of the person 30. If an improper sitting position is detected, an alert (audible and/or visible) or tactile can be output.

According to an embodiment, a network 10 with a plurality of network devices 100 implemented as lighting or luminar devices can be provided. The network devices 100 comprise microphones (sound sensors 130) and/or speakers (sound generators 120).

Therefore, the sound generators 120 and the sound sensors 130 can be distributed among a sensing area 20.

The sitting position detector 200 can be implemented as a dedicated device or can be integrated in a wireless hub or central control unit. Alternatively, the sitting position detection 200 may also be performed by one of the network devices 100. Alternatively, the sitting position detection may be performed by a remote device like a server in the cloud. Furthermore, the sitting position detection may be performed by an internet-of-things IoT edge device. Therefore, the position where the sitting position detection is performed is not relevant as long as the function is performed based on the audio signals detected by the sound sensors of the network devices.

Moreover, the sitting position detection may also be performed by a smart device which can be positioned in the sensing area and which can communicate with the network 10 and the network devices. As an example, an application may be running on such a smart device which is performing the sitting position detection. Alternatively, the sitting position detection may be performed by a smart speaker or a smart watch which can be arranged in the sensing area.

According to an embodiment, several network devices 100 are arranged in the sensing area 20. A first plurality of network devices 100 may be arranged in the vicinity of a person 30. A second plurality of network devices 100 may be arranged further away from the first plurality of network devices. In order to improve the accuracy of the sitting position detecting, the sitting position detector 200 may select some of the first plurality of network devices for being used in the first sensing channel (e.g. inaudible sound signals). Moreover, the frequencies of the inaudible signal as well as the type of audible signal which is generated and is transmitted can be selected by the sitting position detector 200. The sitting position detector 200 may consider a distance between the network devices 100 (signal generator; sound sensors) to the person as well as an impact of any disturbances in the sensing area on the sensing signal. Furthermore, any disturbances from further persons in the vicinity of the first person may be detected. The frequency of the sensing signal may be adapted if required.

Furthermore, optionally, according to the distance between the network device and the chair or person, the sensing frequencies may be selected. In particular, the longer the distance between the network device and the chair, the lower the frequency of the sensing signal. If a received signal strength at the sound detector is not sufficient, another network device in the network may be selected for generating and/or detecting the sensing signal. Moreover, the network device (speaker; microphone) can be selected based on the CSI.

Furthermore, the sitting position detector 200 may pause or interrupt a sitting position detection if a further person or object is determined as moving in the sensing area. Once the disturbance has been removed, the sitting position detection can be resumed. In a further embodiment, the sitting position detection can be used for detecting a health condition for example of elderly people. The sitting position detector 200 can be determined as described above. If a slump posture is detected, this may indicate that the person has a depression. In such a situation, the sitting position detector may issue an alert to the person to motivate the person to sit upright. According to this embodiment, the sitting position of elderly persons is monitored based on audio sensing (as described above).

According to an embodiment, the sitting position detector 200 may track the sitting position of the person and may determine if a person recently started to lean more towards the left or right or if a sudden decrease in sitting position stability is detected, this may indicate a recent muscular or spinal injury. A corresponding alert may be issued.

According to a further embodiment, the sitting position detection may also be used for fall detection. In particular, the sitting position detector may detect when a person sitting on the chair is falling from the chair. The audio sensing of the person can analyze the pressure points (hip position) and may detect a fall for example of an elderly person onto the table surface or to the ground.

According to a further embodiment, the information from the sitting position detection may be used during an RF position or motion sensing. Radio frequency-based sensing is a sensing mechanism involving wireless transceivers (or transmitters/receivers) arranged for transmitting and receiving radiofrequency (RF) signals. These RF signals, which may also be used for radio communication, when passing through a sensing volume, are affected by presence/movement of a person within the sensing volume e.g., via reflection, absorption, scattering etc. The radiofrequency-based sensing uses such deviations of radiofrequency signals to infer presence/motion of the person. Radiofrequency-based sensing also extends to other applications such as location detection, fall detection, gesture detection, vital signs detection etc. which are also based on how radiofrequency signals are affected in the sensing volume.

The network devices 100 may be configured to communicate with each other based on RF signals. In an example, these RF signals may be used for for radio frequency-based sensing. Alternative to using network devices 100 for RF-based sensing, other netowrk devices may be used for RF-based sensing. In an example, the presence of the subjects (30) in the sensing area 20 may be determined via RF-based sensing.

In particular, the knowledge of the body posture may be used to improve the capture of breathing and heartrate related movements of the body. For example, the location of the network devices 100 (or other devices) for RF-based sensing may be selected based on the sensing outcome of the audio sensing, e.g., from the outcome of the sitting position detector 200. In other words, the selection of the location of the network device 100 may be based on the sitting position of the subject 30. In an example, the network devices 100 in proximity with to or a have a direct line of sight with subjects chest/body may be selected for breathing and other heartrate related movement of the subject 30. Therefore, after the audio based sensing of a sitting position detection is performed, this information may be used for the RF motion sensing. The RF motion sensing may be performed by the network devices 100. Here, any RF signals transmitted by a first network device 100 and received by a second network device 100 may be analyzed in view of a motion of a person. Hence, a combination of audio and RF-based sensing is provided.

According to an embodiment, if based on the sitting position detection it is detected that a person has fallen from the chair, the sensing arrangement may be reconfigured and those network devices are chosen which have a direct line of sight to the chest area of the person which might have fallen from the chair in order to more accurately measure the vital signs of the person. The vital signs measurement can be performed based on audio sensing or RF sensing. A slouching posture leaning towards the table may indicate a stroke so checking the breath rate and/or heart rate of such a person can be performed.

In FIG. 3A, a network device 100 is placed on the desk and can be implemented as a table lamp (the network device may also comprise a light unit 140). The sound generator 120 of this network device may emit an audible sound for example between 20 Hz and 15 KHz. The sound generator 120 can also be able to generate inaudible sound. Any network devices 100 arranged at the ceiling may also comprise lighting units 140.

In FIG. 3B, the ceiling network devices 100 may also comprise a speaker 120 which is able to emit and receive audible sound for example between 20 Hz and 15 KHz. The sound generator 120 may also be able to generate inaudible sound.

In the embodiment of FIG. 3C, the network device 100 may be placed on a table 400 and may comprise a sound generator 120 and a sound sensor 130. The frequency at which this sound generator generates and emits sound is in the inaudible sound range, namely for example 18 KHz-20 KHz. This network device may also comprise a sound sensor 130 for receiving the multipath signals after an interaction with the person 30. The respective sensing signal can be used to detect a healthy sitting posture or position or an improper sitting position. Here, the 3D positions (X, Y, Z) of the head 31 as well as the body pressure points (hips) 32 on the chair can be used as indicators whether or not the sitting position is healthy or not. If based on a typical lumbar angle of 20° (the lumbar angle describes how much the body is deviating from the center axis) as shown in FIG. 1 and assuming a typical length of a human torso plus head of about 75 cm, the maximum deviation of the head position from the center axis is only allowed to be between 25 cm to constitute a healthy sitting posture. Accordingly, the detection accuracy must be below 25 cm.

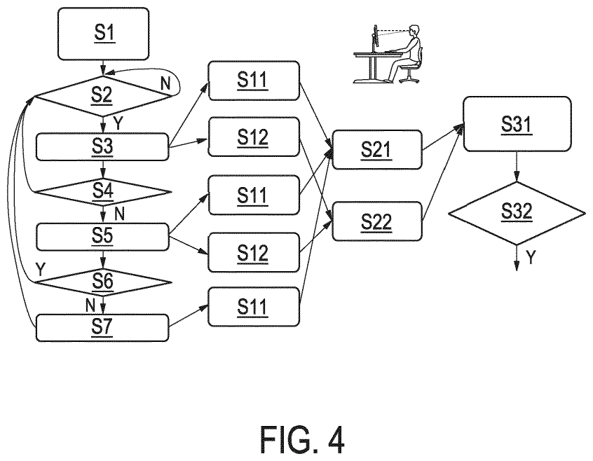

FIG. 4 shows a flow chart of a method of sound based sensing of a person in a sensing area. In step S1, the initialization is started. In a system for example as described with reference to FIG. 1, several network devices 100 can be implemented as ceiling based network devices. The network devices 100 may comprise a sound generator 120 and sound sensors 130. A sound signal is emitted by at least one of the sound generators 120 and the reflected audio signal is detected by at least one of the sound sensors 130. Based on the reflected sound signal, a position of a desk 40 in a sensing area 20 is determined. This can for example be performed by acoustic imaging analyzing the distance and arrival angles of the reflected signal from the desk surface.

Alternatively, the desk position in the sensing area 20 may also be input manually or the information of the desk position may be obtained by RF-based sensing, for example performed by the network devices 100.

Furthermore, in step S1, in addition to determining a position and orientation of a desk, the position and orientation of a chair or desk chair on which the person is sitting or will sit down is determined. If a plurality of network devices is arranged in the sensing area 20, the sitting position detector 200 can select some of these network devices 100. Preferably, at least one of the network devices is selected which has a line of sight towards the chair. This is advantageous as it can be avoided that other network devices which are not in a line of sigh towards the chair may send audio signals which are however blocked by a desk or the desk surface. Preferably, several network devices 100 can be chosen for the sitting position detection which are arranged at different positions in the sitting area 20. This can allow a greatly improved sitting position accuracy. Preferably, in particular those network devices 100 are chosen from among the plurality of network devices 100 in the sensing area 20 which have a sideways view on the chair which is used by the user. With such a sideways view the audio sensing can have a non-occluded view on the pressure points on the chair, i.e. the hip position. Optionally, the orientation of the back rest of the chair is determined and then the respective network devices which are optimally suited for an audio sensing direction parallel to the back rest is selected.

In step S2, a human presence can be detected. If no human presence is detected, then the human presence detection is repeated. If a human is detected, the flow can continue to step S3. Here, a network device 100 on or at a desk (for example in form of a table or desk lamp can be used. The sitting position detector 200 (or the controller 210) controls this network device 100 on or at the table to generate and emit a first audible audio signal on the first audio channel. The frequency of this waveform is preferably in the audible range. The sound sensors 130 of the network devices 100 in or at the sensing area detect audio multipath signals which have interacted with the chair, the desk and the person 30 sitting at the desk. Optionally, a number of sound sensors 130 can be activated to receive the multipath signals. Thus, a microphone array can be achieved for detecting the audio sensing signal.

The sitting position detector 200 can perform an audio sensing algorithm determining the head position and/or detecting pressure points of the person on the chair surface (i.e. the hip position).

In step S4, the accuracy of the first sensing signal is determined. If the accuracy is not sufficient, the network devices 100 arranged in or at a ceiling in the sensing area can be activated to emit a second sensing signal with audible waveforms. Hence, it is determined whether or not the first sensing signal in the first audio sensing channel allows an accurate estimation of the hip position. It should be noted that due to the presence of the desk surface, the detection accuracy based on the first sensing signal (with the audible waveform) may not be sufficient to reliable detect the pressure points of the person (i.e. the hip position). Moreover, it is also possible that the desk surface reflects some of the first sensing signals from the network device 100 placed in or at the desk such that the audio signal received by the sound sensors 130 in the network devices (for example arranged at the ceiling) may be too weak for an accurate and reliable detection.

The sitting position detector 200 can compare the position estimated based on the first audio sensing channel (first audible waveform from the desk network device) with the position estimation from a second audio signal emitted by the (ceiling) network devices. If the two position estimation agree sufficiently well, in step S6, the position estimation based on the first audio sensing signal can be selected. In such a case, only the sensing signals from the (desk) network device 100 can be selected. Thus, it is not necessary that the (ceiling) network device emits a sound. This is advantageous as it will reduce the noise generated by the sitting position detection.

According to an embodiment, in a first step, the (desktop) network device 100 emits an inaudible waveform to detect a position of the person 30 sitting at the desk. If the signal strength from this first sensing step is sufficiently good, the sitting position detector will estimate the sitting position of the user based on the signal. However, if the signal strength is not sufficient, the sitting position detector 200 can control the (ceiling) network devices to emit a second sensing signal (audible signal). Alternatively, the sitting position detector 200 may control the (desktop) network device 100 to emit an audible audio sensing signal.

According to an embodiment, the sitting position detector 200 controls a number of network devices 100 arranged in the sensing area 20 (for example at the ceiling) to perform the sitting position detection. Preferably, at least one sound generator 120 of these network devices 100 generates a sound waveform in the audible range. The reflected sound is detected by at least one of the sound sensors 130 of at least one of the network devices 100 and the sitting position detector 200 determines for example a head position of a user. Moreover, the reflected audio signals are analyzed to determine a position of a chair, a table and the person 30. Here, preferably the audio waveform is in the audible frequency range as the network devices 100 will be arranged at a distance of more than 2 m away from the person. The sitting position detector 200 analyzes the reflected audio signals to determine the position of for example the head position and a hip position of a user. If this first analyzing step does not lead to a sufficient accuracy, the sitting position detector may control the sound generators 120 in the network devices 100 to emit a second audio waveform for example in the inaudible frequency range. Then again the sitting position detector analyzes the reflected sound signals to determine the position of the person in view of an improper sitting position. This two step approach is advantageous as it will reduce the amount of ultrasonic sound generated by the network devices. Thus, the ultrasonic noise can be reduced in a sensing area 20.

According to an embodiment, the sound waveform in the audible frequency range can be concealed in a white noise emitted by a noise making device (which can be for example be utilized to suppress intangible speech disturbance in the office).

According to an embodiment, the sound generators 120 of at least one of the network devices 100 in the sensing area 20 may generate an audio signal which is designed for suppressing audible speech disturbances in offices. This audio signals can be adapted to mask any of the audio signals generated by the network devices for the audio sensing purpose. This is advantageous as a person for example working in the sensing area will not be disturbed by the audio based sensing for the sitting position detection.

Alternatively, the audio sensing can also be embedded in a public service announcement or in a music reproduction. This can be advantageous as it will allow a reduction of a disturbance of persons 30 in the sensing area 20 by the audio based sensing. According to an embodiment, the audio based sensing can for example be performed parallel to the reproduction of an announcement message or when a music is reproduced. Accordingly, the audio based sensing can be unobtrusively embedded in any announcement or music.

According to an embodiment, the network device in or at the table (for example implemented as a table lamp) is able to emit an inaudible audio waveform (>18 KHz) which can be used for the head position estimation. Preferably, the network device 100 on the desk should be arranged at a distance of 50 to 80 cm to achieve a good position estimation.

In step S21 and step S22, a head position and a chair and surface position can be fused based on the head positions and chair surface positions obtained by the first audible audio sensing channel and the second inaudible audio sensing channel. Here, a Bayesian approach may be applied.

According to an embodiment, for example a hidden mark module HMM can be used to track the head and chair surface positions by analyzing historic data for individual persons. If multiple persons are occupying the sensing area, one of the network devices may lead to monitor sitting postures of more than one person. Preferably, a tracking algorithm can be used to effectively reduce an interference of position estimations with the positions of other persons in the sensing area 20.

According to an embodiment, the sitting position detector 200 calculates a relative distance between the head, the location of the pressure points of the body on the chair (hip position) and the touching positions of the arms on the desk surface. A machine learning module can be trained with labelled data stating whether the sitting position is healthy or not. Alternatively, a rule-based algorithm utilizing a distance threshold depending on the lumbar angle, cervical angle, etc. can be used.

According to an embodiment, the audio sensing can also be embedded in an app of a phone or laptop. Optionally, the phone or laptop can act as a network device. Optionally, received audio signals from the sound sensors 130 in the network devices 100 can be used to detect a position of a user. Furthermore, this information can be used to guide a person using the phone or laptop to position the phone or laptop. Here, the phone or laptop can emit audio waveforms (in the audible or inaudible frequency range). The reflected signals can then be received by the sound sensors 130 in the network devices to enable a position detection. Alternatively, the smart device or laptop may comprise microphones which can be used as sound sensors to detect reflected audio signals. The signals can then be used by the sitting position detector to detect a sitting position of a person. Accordingly, a smart device or a laptop can function as a network device as described above.

Assuming the network has M speakers and N microphones, the received signals are

$$[ \begin{matrix}{y}_{0} ( t ) \\ … \\ {y}_{N - 1} ( t )\end{matrix} ] = [ \begin{matrix}{h}_{0 , 0} & … & {h}_{0 , M - 1} \\ … & … & … \\ {h}_{N - 1 , 0} & … & {h}_{N - 1 , M - 1}\end{matrix} ] [ \begin{matrix}{a}_{0} {x}_{0} ( t ) \\ … \\ {a}_{M - 1} {x}_{M - 1} ( t )\end{matrix} ] + [ \begin{matrix}{n}_{0} ( t ) \\ … \\ {n}_{M - 1} ( t )\end{matrix} ] \qquad ( 1 )$$

Where yn(t) is the received signal at the nth microphone, xm(t) is the unit transmission signal from the mth speaker, am is the transmission gain, nm(t) is the noise at the microphone, hn,m(t) is the channel response for the signal from the mth speaker to the nth microphone, where arrival angles of different paths are embedded in hn,m(t). It is assumed that microphone arrays are used at each node, then we have received signal at the pth element sensor,

$${y}_{n , p} ( t ) = cos [ - \frac{2 π Δ c o s θ}{λ} p + \frac{4 π Bd}{T {V}_{s}} t + ϕ ] \qquad ( 2 )$$

where, θ is the AoA, and dis the distance, Δ, λ, B, T, and vₛ are the MIC separation, wavelength, bandwidth, chirp length, and sound propagation speed, respectively. ϕ is a constant phase term. We see that the received signal is a 2D sinusoid with frequencies 2πΔcosθ/λ.

In time domain, according to an embodiment, orthogonal audio pulses (like direct-sequence spread spectrum) are designed for each speaker so the speakers will cause no or minimum neglectable interference to each other and also to itself, for example the delayed signals due to multiple reflections. The identity of the signal received at the network device (a luminaire-based microphone) (i.e. did the signal originate from speaker 1, or 2, or a combination of 1 and 2 can be detected). The microphone could use an orthogonal matched filter to filter out the signals for the sound generators of the network 1 and 2, respectively. Hence a time beamforming can be achieved.

According to an embodiment, it is proposed to preferably use a lighting system embedded with a multitude of microphone sensors as detecting units distributed across the room to monitor a subject status and/or position. The sound generator can also be integrated within a subset of the lighting fixtures. For instance, very affordable ultra-cheap audio-transmission elements, which are capable of sending just one beep at a pre-selected fixed frequency, are readily available from children's toys at very low cost and can be utilized as sound generator. If a more advanced programmable audio frequency as predetermined sound is desired for further improving the audio sensing performance, a range of suitable, very affordable programmable speaker products are available that can be used as sound generator.

According to an embodiment, the network devices may be part of a lighting system and can each comprise at least one light unit. The network devices may furthermore include a speaker (sound generator) and a microphone (sound sensor). In particular, the network device can be adapted to conduct the audio sensing via at least two different audio sensing channels to detect a sitting posture of a user or a subject. Optionally, a first audio sensing channel may relate to a first short range audio sensing and the second audio sensing channel may relate to a second long range audio sensing channel formed by multiple lighting embedded speakers and microphones. Advantageously, a combination of two different audio sensing channels (a first channel to determine a head position and a second channel to determine a hip position) will significantly improve the accuracy of the audio sensing.

According to an embodiment, the waveform of one sound generator in an audio sensing channel can be controlled. However, preferably, two different audio sensing channels each with a different frequency can be used. For example, to detect a head position, a first audible sound emitted from a ceiling light and a second inaudible sound for example from a table lamp can be used to achieve a high accuracy for the head detection.

According to an embodiment, a number of network devices (sensing notes) can be provided. Here, a network device may comprise a sound generator and/or a sound sensor. Optionally, the sound sensor and/or the sound generator can be operated in two different audio channels. Preferably, two pairs of nodes can be assigned for the first audio sensing channel and the second audio sensing channel.

According to an embodiment, in an environment with a plurality of speakers and microphones (sound generators; sound sensors) which can be close to for example a table or desk, the best selection of frequencies and used sensing nodes may be determined. In particular, the distance between the microphone and the speaker to the chair as well as the impact from disturbances on the sensing signal from body movement by a second person in the vicinity may be considered when choosing the respective sound generators and sound sensors. In particular, the longer the distance to the chair where the person is sitting, the lower the sensing frequency should be adapted. If the received signal strength at the microphone (sound sensor) is insufficient for example if a low sensing frequency has been chosen, another speaker and/or microphone can be assigned to perform the posture detection.

According to an embodiment, not only the absolute sitting posture of a person is detected, but rather abnormalities of a sitting posture (compared to usual sitting postures) may be detected. This can be used as a sign of an health issue, as fatigue detection in cars, trains, busses, for fatigue detection or accident prevention at a workshop or a factory. To avoid the above mentioned drawbacks of the prior art, it its proposed in this invention, inter alia, to utilize a distributed microphone grid, i.e. sound detector grid, integrated within luminaires, in order to monitor a sitting position of a person. The proposed audio sensing solution is capable of monitoring the true status of the person.

Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Procedures like the controlling of the sound detector or the sound generator, the providing of the baseline, the determining of the status and/or position of the subject, et cetera, performed by one or several units or devices can be performed by any other number of units or devices. These procedures can be implemented as program code means of a computer program and/or as dedicated hardware.

A computer program may be stored/distributed on a suitable medium, such as an optical storage medium or a solid-state medium, supplied together with or as part of other hardware, but may also be distributed in other forms, such as via the Internet or other wired or wireless telecommunication systems.

Any reference signs in the claims should not be construed as limiting the scope.

1. A system for performing a sound-based sensing of subjects in a sensing area, comprising
a network comprising at least two network devices configured to perform a sound-based sensing in a sensing area,
wherein the network devices are distributed in the sensing area and are configured to communicate with each other based on RF signals,
wherein at least one network device comprises a sound generator configured to generate a sound signal and at least one network device comprises a sound sensor configured to detect the sound signal from the sound generator after a propagation through at least a portion of the sensing area and to generate a sensing signal indicative of the detected sound signal, wherein the sound generator and the sound sensor are arranged in different network devices, wherein the sound generator is configured to generate the sound signal with frequencies which can vary in time or to generate different for the different network device, and wherein the generation of different frequencies is based on the position of the network devices in the sensing area, and
a sitting position detector configured to detect a sitting position of the subject in the sensing area based on the sensing signal from the sound sensor, wherein the sitting position is a hip position and/or a head position of the subject sitting at a table or desk in the sensing area.

2. (canceled)

3. The system according to claim 1, wherein
the sitting position detector comprises a controller which is configured to control the operation of at least one of the network devices to perform the sound-based sensing in the sensing area.

4. The system according to claim 3, wherein
the controller is configured to control the operation of at least one sound generator to determine a sound waveform and/or a frequency of the generated sound signal in at least a first and/or a second audio channel.

5. The system according to claim 4, wherein
the first audio channel is in an audible frequency range and the second audio channel is in an inaudible frequency range.

6. The system according to claim 4, wherein
at least one sound generator is configured to generate a sound signal in a first audio channel,
at least one sound generator is configured to generate a sound signal in a second audio channel,
at least one sound sensor is configured to detect the sound signal in the first audio channel and to generate a sensing signal indicative of the detected sound signal,
at least one sound sensor is configured to detect the sound signal in the second audio channel and to generate a sensing signal indicative of the detected sound signal.

7. The system according to claim 6, wherein
the controller is configured to control one of the sound generators generating an audible sound signal or an inaudible sound signal in the first audio channel and to control one of the sound generator generating the inaudible sound signal or the audible sound signal in the second audio channel,
wherein the controller is configured to initiate the generation of the inaudible sound signal or the audible sound signal only when the sitting position detector has detected a correct head position of the subject.

8. The system according to claim 1, wherein
the sitting position detector comprises a fall detector configured to determine that the subject has fallen off a chair based on the sensing signal from the sound sensor.

9. The system according to claim 1, wherein
the sitting position detector is configured to determine an abnormality regarding a sitting posture compared to a previously determined sitting posture based on the sensing signal from the at least one sound sensor and to issue a warning if an abnormality is determined.

10. The system according to claim 1, wherein
at least one of the network devices comprises a lighting functionality.

11. The system according to claim 1, wherein
at least one network device is implemented as a light unit, in particular a celling lamp, a lighting wall switch or a desk lamp.

12. The system according to claim 1, wherein
at least one network device is implemented as a smart device, smart speaker, or a laptop.

13. The system according to claim 1, wherein
one of the network devices is configured to output or display instructions to a user or how to best position the sound generator and/or the sound sensor.

14. A method for performing a sound-based sensing of subjects in a sensing area, based on a network comprising at least two network devices configured to perform a sound-based sensing in a sensing area, wherein the network devices are distributed in the sensing area and are configured to communicate with each other based on RF signals, comprising the steps of:
generating a sound signal by at least one sound generator in one of the network devices, wherein the sound generator is configured to generate the sound signal with frequencies which can vary in time or to generate for the different network device, and wherein the generation of different frequencies is based on the position of the network devices in the sensing area,
detecting the sound signal from the at least one sound generator in one of the network devices after a propagation through at least a portion of the sensing area
generating a sensing signal indicative of the detected sound by a sound sensor in one of the network devices,
wherein the sound generator and the sound sensor are arranged in different network devices, and
detecting a sitting position of a subject in the sensing area based on the sensing signal from the sound sensor, wherein the sitting position is a hip position and/or a head position of the subject sitting at a table or desk in a sensing area.

15. A computer program product for performing a sound-based sensing of a subject in a sensing area, wherein the computer program product comprises program code means for causing the system of claim 1.
